(19) United States
(12) Patent Application Publication
(10) Pub. No.: US 2007/0219428 A1
(43) Pub. Date: Sep. 20, 2007

(54) Method of providing a floating life-sustaining facility

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/531,651

(22) Filed: Sep. 13, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.
Application No. 11/466,727 is a continuation-in-part of application No. 11/466,953, filed on Aug. 24, 2006.
Application No. 11/466,953 is a continuation-in-part of application No. 11/470,156, filed on Sep. 5, 2006.
(60) Provisional application No. 60/823,806, filed on Aug. 29, 2006.

(51) Int. Cl. A61B 5/00

(52) U.S. Cl. 600/300

(57) ABSTRACT

A floating life-sustaining facility (205) is provided (105), and consideration-based private civil security subscriptions are accepted (110) from subscribers with respect to selectively providing benefits for authorized beneficiaries with respect to the floating life-sustaining facility (205) in event of a civilly-catastrophic event. A variety of features and/or survival items may be provided in association with the floating life-sustaining facility (205), and access to the facility and/or survival items may be selectively provided for authorized beneficiaries of the subscriptions based at least in part on the type of consideration-based private civil security subscription possessed by the authorized beneficiary.

### RELATED APPLICATIONS

This application claims the benefit of U.S. Provisional Application No. 60/823,806, filed Aug. 29, 2006, the contents of each of which are fully incorporated herein by this reference.

This application comprises a continuation-in-part of:

SUBSCRIPTION-BASED PRIVATE CIVIL SECURITY FACILITATION METHOD AS FILED ON Mar. 17, 2006 AND HAVING application Ser. No. 11/384,037;

SUBSCRIPTION-BASED CATASTROPHE-TRIGGERED MEDICAL SERVICES FACILITATION METHOD AS FILED ON Mar. 30, 2006 AND HAVING application Ser. No. 11/394,350;

PERSONAL PROFILE-BASED PRIVATE CIVIL SECURITY SUBSCRIPTION METHOD AS FILED ON Apr. 11, 2006 AND HAVING application Ser. No. 11/279,333;

RADIATION SHELTER KIT APPARATUS AND METHOD AS FILED ON Apr. 24, 2006 AND HAVING application Ser. No. 11/379,929;

FRACTIONALLY-POSSESSED UNDERGROUND SHELTER METHOD AND APPARATUS AS FILED ON May 2, 2006 AND HAVING application Ser. No. 11/381,247;

SUBSCRIPTION-BASED CATASTROPHE-TRIGGERED TRANSPORT SERVICES FACILITATION METHOD AND APPARATUS AS FILED ON May 2, 2006 AND HAVING application Ser. No. 11/381,257;

SUBSCRIPTION-BASED MULTI-PERSON EMERGENCY SHELTER METHOD AS FILED ON May 2, 2006 AND HAVING application Ser. No. 11/381,265;

SUBSCRIPTION-BASED CATASTROPHE-TRIGGERED RESCUE SERVICES FACILITATION METHOD AND APPARATUS AS FILED ON May 2, 2006 AND HAVING application Ser. No. 11/381,277;

DOCUMENT-BASED CIVILLY-CATASTROPHIC EVENT PERSONAL ACTION GUIDE FACILITATION METHOD AS FILED ON May 12, 2006 AND HAVING application Ser. No. 11/383,022;

RESCUE CONTAINER METHOD AND APPARATUS AS FILED ON May 26, 2006 AND HAVING application Ser. No. 11/420,594;

PURCHASE OPTION-BASED EMERGENCY SUPPLIES PROVISIONING METHOD AS FILED ON Jun. 1, 2006 AND HAVING application Ser. No. 11/421,694;

SUBSCRIPTION-BASED PRE-PROVISIONED TOWABLE UNIT FACILITATION METHOD AS FILED ON Jun. 12, 2006 AND HAVING application Ser. No. 11/423,594;

RADIATION-BLOCKING BLADDER APPARATUS AND METHOD AS FILED ON Jun. 19, 2006 AND HAVING application Ser. No. 11/425,043; AND

PRIVATE CIVIL DEFENSE-THEMED TELEVISION BROADCASTING METHOD AS FILED ON Jun. 23, 2006 AND HAVING application Ser. No. 11/426,231;

EMERGENCY SUPPLIES PRE-POSITIONING AND ACCESS CONTROL METHOD AS FILED ON Jul. 10, 2006 AND HAVING application Ser. No. 11/456,472;

PRIVATE CIVIL DEFENSE-THEMED BROADCASTING METHOD AS FILED ON Aug. 1, 2006 AND HAVING application Ser. No. 11/461,605; AND

METHOD OF PROVIDING VARIABLE SUBSCRIPTION-BASED ACCESS TO AN EMERGENCY SHELTER AS FILED ON Aug. 1, 2006 AND HAVING application Ser. No. 11/461,624;

SUBSCRIPTION-BASED INTERMEDIATE SHORT-TERM EMERGENCY SHELTER METHOD AS FILED ON Aug. 7, 2006 AND HAVING application Ser. No. 11/462,795;

SUBSCRIPTION-BASED CATASTROPHE-TRIGGERED RESCUE SERVICES FACILITATION METHOD USING WIRELESS LOCATION INFORMATION AS FILED ON Aug. 7, 2006 AND HAVING application Ser. No. 11/462,845;

PRIVATELY PROVISIONED SURVIVAL SUPPLIES DELIVERY METHOD AS FILED ON Aug. 15, 2006 AND HAVING application Ser. No. 11/464;751;

PRIVATELY PROVISIONED SURVIVAL SUPPLIES SUB-UNIT-BASED DELIVERY METHOD AS FILED ON Aug. 15, 2006 AND HAVING application Ser. No. 11/464,764;

PRIVATELY PROVISIONED SURVIVAL SUPPLIES ACQUISITION METHOD AS FILED ON Aug. 15, 2006 AND HAVING application Ser. No. 11/464,775;

PRIVATELY PROVISIONED SURVIVAL SUPPLIES CONTENT ACQUISITION METHOD AS FILED ON Aug. 15, 2006 AND HAVING application Ser. No. 11/464,788;

METHOD TO PRIVATELY PROVISION SURVIVAL SUPPLIES THAT INCLUDE THIRD PARTY ITEMS AS FILED ON Aug. 15, 2006 AND HAVING application Ser. No. 11/464,799;

WASTE DISPOSAL DEVICE AS FILED ON Aug. 16, 2006 AND HAVING application Ser. No. 11/465,063;

SUBSCRIPTION-BASED PRIVATE CIVIL SECURITY RESOURCE CUSTOMIZATION METHOD AS FILED ON Aug. 23, 2006 AND HAVING application Ser. No. 11/466,727;

PREMIUM-BASED PRIVATE CIVIL SECURITY POLICY METHODS AS FILED ON Aug. 24, 2006 AND HAVING application Ser. No. 11/466,953;

SUBSCRIPTION-BASED MOBILE SHELTER METHOD AS FILED ON Sep. 5, 2006 AND HAVING application Ser. No. 11/470,156; the contents of each of which are fully incorporated herein by this reference.

### TECHNICAL FIELD

This invention relates generally to providing access to emergency shelters and provisions.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related interactions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swatch of a given civil community. Any number of natural and/or non-naturally-caused events can sufficiently disrupt society's infrastructure and ability to provide one or more life-sustaining resources such as water, nutrition, shelter, and the like.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a civilly-catastrophic event occurs.

Such insights, of course, are not particularly new. Civil preparedness shortcomings occasionally attract public attention, and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk survival (let alone the comfort) of their citizens in the face of most civilly-catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

### BRIEF DESCRIPTION OF THE DRAWINGS

The above needs are at least partially met through provision of the method of providing a floating life-sustaining facility as described in the following detailed description in conjunction with the drawings, wherein:

FIG. 1 comprises a flow diagram as configured in accordance with various embodiments of the invention;

FIG. 2 comprises an elevation and partially cut-away view of example floating life-sustaining facilities as provided in accordance with various embodiments of the invention;

FIG. 3 comprises a perspective view of an example floating life-sustaining facility as provided in accordance with various embodiments of the invention;

FIG. 4 comprises a flow diagram of optional features of a selectively submergible chamber as provided in accordance with various embodiments of the invention; and

FIG. 5 comprises a flow diagram of optional features of a floating life-sustaining facility as provided in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a floating life-sustaining facility is provided, and consideration-based private civil security subscriptions are accepted from subscribers with respect to selectively providing benefits for authorized beneficiaries with respect to the floating life-sustaining facility in event of a catastrophic event. A variety of features and/or survival items may be provided in association with the floating life-sustaining facility, and access to the facility and/or survival items may be selectively provided for authorized beneficiaries of the subscriptions based at least in part on the type of consideration-based private civil security subscription possessed by the authorized beneficiary.

The nature of the floating life-sustaining facility can vary. For instance, a floating life-sustaining facility may include a buoy with a compartment for storing life-sustaining resources or a water craft to use as a secure emergency shelter for authorized beneficiaries. In this way, the provider of the facilities can provide varying levels of comfort, convenience, and/or security to authorized beneficiaries of such consideration-based private civil security subscriptions. Optionally, the floating life-sustaining facilities may be movable such that the facilities may be pre-positioned according to relative need and/or a likelihood that the provision of the facility or resources stored on the facility may be compromised by nature, human, or other interference. Thus, providers of such facilities may be able to provide more predictable and reliable access to a pre-provisioned secure emergency shelter upon the occurrence of a civilly-catastrophic event.

These steps are facilitated without dependency upon governmental oversight, participation, or control. The particular resources provided at the floating life-sustaining facility can vary with the needs and requirements of the authorized beneficiaries. Importantly, an individual can take important steps to bring a considerably improved measure of security into their lives without having to effectively become a full-time survivalist; such individuals can, in short, continue to enjoy their chosen vocations and standard of living knowing that, should a civilly-catastrophic event indeed be visited upon them, they will have extraordinary access to one or more resources that are critical to human life.

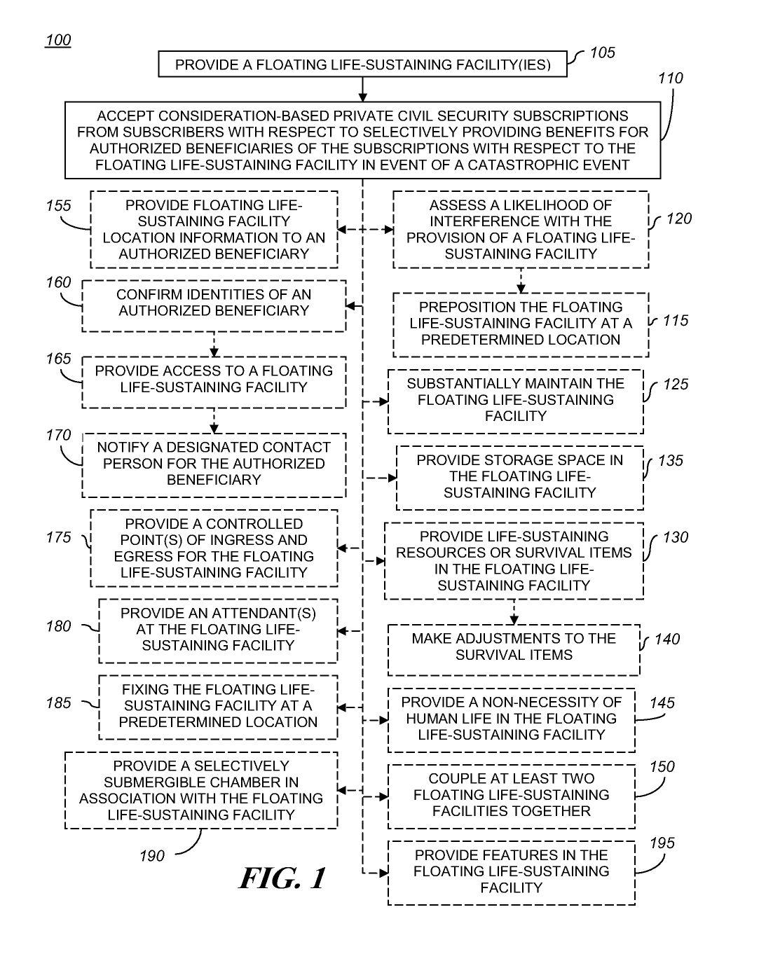

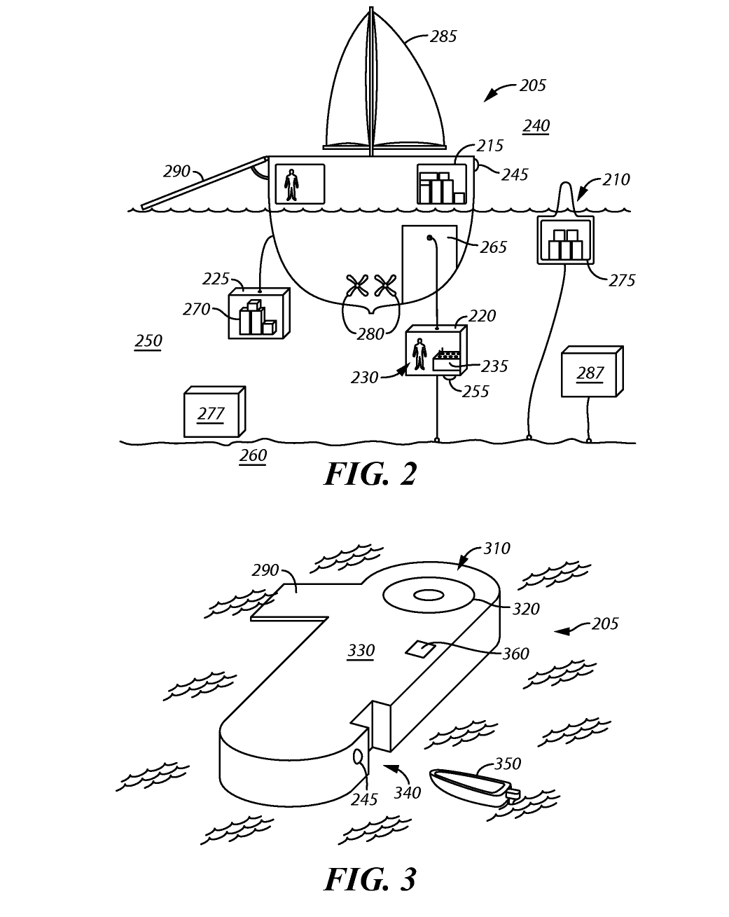

These and other benefits may become clearer upon making a thorough review and study of the following detailed description. Referring now to FIG. 1, an illustrative process 100 includes providing 105 a floating life-sustaining facility and accepting 110 consideration-based private civil security subscriptions from subscribers with respect to selectively providing benefits for authorized beneficiaries of the subscriptions with respect to the floating life-sustaining facility in event of a civilly-catastrophic event. With brief reference to FIG. 2, the floating life-sustaining facility may include a floating shelter 205 and/or a floating store of life-sustaining resources 210. By one approach, a floating shelter 205 may also include storage 215. Pursuant to these teachings, a floating life-sustaining facility may comprise a secure emergency shelter designed to protect one person, that is mobile or immobile, or that may be a secure multi-person emergency shelter. (Despite these many possible variations, for convenience, any of a variety of floating life-sustaining facilities will be referred to generally by reference number 205, except as specifically noted otherwise herein.)

It is contemplated that a floating life-sustaining facility may be pre-positioned 115 at a predetermined location. Then, when pre-positioning the floating life-sustaining facility, the provider of the facility may assess 120 a likelihood of interference with the provision of the floating life-sustaining facility. The assessment 120 typically can include assessing the likelihood of the occurrence of at least one particular event, the location of the particular event(s), and the likelihood that the event(s) will interfere with the provision of benefits from the floating life-sustaining facility. For instance, a provider of the floating life-sustaining facility may weigh the likelihood that weather, water conditions, and/or other people may damage or disable the facility. In other words, the provider of the facility will typically weigh these issues in an effort to maintain the facility.

As such, these teachings will accommodate a process 100 that also often includes substantially maintaining 125 the floating life-sustaining facility. Maintaining 125 the floating life-sustaining facility may include maintaining the floating life-sustaining facility pending a need to permit subscription-based access to the floating life-sustaining facility upon occurrence of the civilly-catastrophic event. In such a case, the floating life-sustaining facility may be more consistently and readily available for immediate occupation by authorized beneficiaries in a time of need. Optionally, a provider may maintain 125 the floating life-sustaining facility on station at least twelve miles away from any coast line of a sovereign entity. Similarly, a provider may maintain 125 the floating life-sustaining facility on station in international waters outside the jurisdiction of any one sovereign nation. In such situations, the floating life-sustaining facility is less likely to be subject to local or national laws and/or be commandeered by such sovereignties in times of need, thereby increasing the reliability of the availability of the floating life-sustaining facility for authorized beneficiaries. Thus, it is advantageous to provide a floating life-sustaining facility that is movable to and maintainable at a position outside of national territorial waters.

The process 100 may also include providing 130 life-sustaining resources or survival items in the floating life-sustaining facility. For instance, the process 100 may include providing 135 storage space in the floating life-sustaining facility, in which the life-sustaining resources and/or survival items may be stored. Also, authorized beneficiaries may store personal items in the storage space. From time to time, the provider of the floating life-sustaining facility may make adjustments 140 to the survival items in the facility according to the needs or wishes of the authorized beneficiaries of subscribers. As such, the provider may provide a plurality of life-sustaining resources as pertain to a plurality of differing categories of life's necessities. Non-limiting examples of such life-sustaining resources include at least one of the group including hydration resources, nourishment resources, shelter, clothing, security, medical treatment, and environmentally borne threat abatement.

Similarly, survival items may include consumable survival supplies and/or non-consumable survival supplies. Non-limiting example consumable survival supplies include one or more of food supplies, potable water, finite air supplies, emergency medical supplies, batteries, ammunition, hygiene supplies, sanitation supplies, consumable fishing supplies, signal flares, sunscreen lotion, anti-motion sickness medication, insect repellant, medications, reverse osmosis water filters, supplemental vehicular fuel supplies, and spare parts for the floating life-sustaining facility such as engine parts, a propeller, a bilge pump, and the like. Non-limiting example non-consumable survival supplies include one or more of essential clothing items, personal protection items, bed linens, beds, food preparation items, communications items, power generation equipment, repair and maintenance tools, evacuation tools, navigation equipment, rescue tools and supplies, radiation shielding, biological/chemical agent protection, spare vehicle parts, self-rescue gear, vehicular maintenance tools, sails, signal mirrors, signal flags, life rafts, life boats, rope, life preservers, fishing supplies, vision enhancement devices such as binocular and the like, semaphore materials, deck shoes, and weapons.

By another approach, at least one non-necessity of human life may be provided 145 in the floating life-sustaining facility. Such items may provide an improvement in the quality of life of authorized beneficiaries having access to the facility. Non-limiting examples of such non-necessities of human life include a luxury consumable item; a luxury non-consumable item; non-human nourishment; barter medium; an entertainment apparatus; an educational tool; physical conditioning, exercise, and maintenance training and equipment; crafts supplies and training; and sports equipment and facilities.

It is contemplated that the provider of the floating life-sustaining facility may provide a plurality of floating life-sustaining facilities. In such a situation, at least two floating life-sustaining facilities may be physically coupled 150 together. For instance, at least two floating life-sustaining facilities may be physically coupled together such that a self-powered vehicle, for example a tug boat or other powered craft, can simultaneously transport the coupled floating life-sustaining facilities. Such an arrangement can provide additional flexibility in the storage and shelter accommodations for authorized beneficiaries. For example, one floating life-sustaining facility may be utilized as a secure emergency shelter while a second floating life-sustaining facility may be utilized as storage for various supplies. Alternatively, different floating life-sustaining facilities may provide different levels of shelter accommodations for authorized beneficiaries based at least in part on the type of subscription possessed by the authorized beneficiary.

As such, the coupled floating life-sustaining facilities may include different benefits, accommodations, and features from one another. Non-limiting examples of the different benefits, accommodations, and features as may exist between two or more coupled floating life-sustaining facilities include a floating shelter having a larger selection of foods available relative to another floating shelter, a floating shelter having a larger amount of authorized beneficiary privacy available relative to another floating shelter, a floating shelter having a larger selection of non-necessities available relative to another floating shelter, a floating shelter having a larger selection of consumable items available relative to another floating shelter, a floating shelter having a larger selection of non-consumable items available relative to another floating shelter, a floating shelter having increased security relative to another floating shelter, a floating shelter having increased communications abilities available relative to another floating shelter, a floating shelter having an increased availability of life sustaining resources relative to another floating shelter, a floating shelter having higher quality living quarters relative to another floating shelter, a floating shelter having a larger availability of health care options available relative to another floating shelter, a floating shelter having a larger availability of educational materials available relative to another floating shelter, and a floating shelter having a higher level of entertainment options available relative to another floating shelter.

Examples of having a larger amount of authorized beneficiary privacy available include having an increased amount of private space including at least one of personal hygiene facilities, personal storage space, personal food preparation facilities, personal external communications facilities, personal entertainment space, personal education space, personal training space, personal workshop space, a personal place to worship, storage for at least one personalized item, and personal meeting facilities. Example personal items may include specialized gear, personal dietary requirements, specialized clothing, specialized safety gear, specialized transport, and personal medicine. Example non-necessities are listed above. Example consumable items may include food supplies, potable water, medical supplies, safety supplies such as masks, filters, and the like, and hygiene supplies.

Examples of having an increased availability of life-sustaining resources include having an increased availability of hydration resources, nourishment resources, shelter, and environmentally borne threat abatement items. Examples of having higher quality living quarters include having high quality sleeping accommodations (such as a deeper or better mattress, higher thread count bedding, additional and/or larger pillows, and so forth), food preparation facilities, worship facilities, personal hygiene facilities, business activities facilities, exercise facilities, spa facilities, and sitting accommodations. Finally, examples of having a higher level of entertainment options available include having a larger selection of videos, improved broadcast reception, a larger selection of board games, having access to live entertainment, having a larger selection of arts and crafts materials, having improved religious-activity related facilities, a larger workshop space, a larger selection of workshop tools, and a larger selection of reading materials.

Optionally, one may provide 155 floating life-sustaining facility location information to an authorized beneficiary. This information may be needed by authorized beneficiaries in the event of a civilly-catastrophic event where an authorized beneficiary may need shelter or life-sustaining resources. Typically, the process 100 may also include confirming 160 the identities of authorized beneficiaries gaining access to the floating life-sustaining facility when providing 165 access to a floating life-sustaining facility. The identities of the authorized beneficiaries may be confirmed using any known method including, for example, the use of biometrics, an identification card, photographs, visual confirmation, an admission card, a password, a pass code, a voice print, and words. Additionally, a designated contact person for an authorized beneficiary may be notified 170 in conjunction with the authorized beneficiary's entry into the floating life-sustaining facility.

Access to a floating life-sustaining facility or portions thereof may thus be limited to authorized beneficiaries or limited according to a type of subscription possessed by an authorized beneficiary. Optionally, then, the floating life-sustaining facility may be provided 175 with a controlled point of ingress and egress to control such passage. Additional controlled points of ingress and egress may also be provided. A controlled point of access may comprise a secured loading dock or pier. By another approach, the point of ingress and egress may include a sealable point of ingress and egress for the floating life-sustaining facility. Such a sealable point of ingress and egress may include a door than opens inwardly and/or a locking mechanism such that access to the floating life-sustaining facility is selectively controllable. An example door or hatch 360 is illustrated in FIG. 3. So configured, the floating life-sustaining facility may be less likely to be contaminated by airborne or other contaminates outside of the floating life-sustaining facility.

A given subscriber may provide a subscription for access to the benefits of a floating life-sustaining facility for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

By one approach, these subscriptions may be accepted by, for example, a for-profit business. By another approach a not-for-profit business (such as a membership-based entity) may be the appropriate entity to offer and accept such subscriptions.

As noted, these teachings provide for a subscription-based approach. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extends past the death of a named subscription beneficiary and further allows for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time-based or event-based duration of access rights or, if desired, when a single payment serves to acquire a perpetual right of access that may be retained, transferred, inherited, or the like);

ownership-based rights of access (as may occur when the subscription provides for ownership rights in the at least one life-sustaining resource);

non-transferable rights of access (as may occur when the subscription, by its terms and conditions, prohibits transfer of the right of access to the at least one life-sustaining resource from a first named beneficiary to another);

transferable rights of access (as may occur when the subscription, by its terms and conditions, permits conditional or unconditional transfer of the right of access to the at least one life-sustaining resource from a first named beneficiary to another);

membership-based rights of access (as may occur when the subscription, by its terms and conditions, establishes a membership interest with respect to the accorded right of access such as, for example, a club-based membership);

fractionally-based rights of access (as may occur when the subscription, by its terms and conditions, establishes a divided or undivided co-ownership interest by and between multiple subscription beneficiaries with respect to a right to access the at least one life-sustaining resource);

non-ownership-based rights of access (as may occur when the subscription, by its terms and conditions, establishes the aforementioned right of access via, for example, a lease, rental, or borrowing construct); and/or

option-based rights of access.

Those accepting subscriptions will typically provide and maintain the floating life-sustaining facilities and the resources provided therewith, thereby incurring significant costs. To adequately scale the costs for the provision and maintenance of the secure emergency shelters, the provider may require the subscribers to acquire different subscriptions, in exchange for different amounts of consideration, for the right to access particular floating life-sustaining facilities or a number of floating life-sustaining facilities.

As used herein, “civilly-catastrophic event” will be understood to refer to an event that substantially and materially disrupts a society's local, regional, and/or national infrastructure and ability to provide in ordinary course for the at least one life-sustaining resource that is the subject of the subscription. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation of that interruption.

A civilly-catastrophic event can be occasioned by any of a wide variety of natural and/or non-natural disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based collisions (as with comets, large asteroids, and so forth), extreme environmental events (such as widespread uncontrolled fire or the like), and global or regional pandemics, to note but a few.

Examples of non-natural disasters capable of initiating a civilly-catastrophic event include both unintended events as well as intentional acts of war, terrorism, madness or the like. Examples of non-natural disasters capable of such potential scale include, but are not limited to, nuclear-related events (including uncontrolled fission or fusion releases, radiation exposure, and so forth), acts of war, the release of deadly or otherwise disruptive biological or chemical agents or creations, and so forth.

To help facilitate the provision of a floating life-sustaining facility, the process 100 may optionally include providing 180 an attendant at the floating life-sustaining facility. In one approach, at least one substantially full-time attendant is provided at the floating life-sustaining facility. As such, the substantially full-time attendant(s) is available to facilitate operation of the floating life-sustaining facility in response to a civilly-catastrophic event. By another approach, at least one attendant is provided at the floating life-sustaining facility upon the occurrence of a civilly-catastrophic event. In this approach, maintenance prior to the civilly-catastrophic event is performed periodically, and the attendant is then available to facilitate the provision of benefits at a time of need.

Typically, the floating life-sustaining facility may comprise a water craft. Non-limiting examples of water craft that may be utilized as a floating life-sustaining facility include a hovercraft, a hydroplane, a hydrofoil, a ship, a submarine, an amphibious vehicle, a barge, a pilotless craft, a single-hulled craft, a double-hulled craft, a triple-hulled craft, a buoy, a tethered storage device, and a remotely piloted craft. Generally, much is known in the art regarding the construction and maintenance of such crafts. Such a construction will usually at least serve to protect the inhabitants or stored goods from environmental stress and extremes. In addition to the hull, deck, and internal structures, these teaching contemplate floating life-sustaining facilities provided for shelter may also include internal temperature control, lighting, storage facilities, sleeping facilities, food preparation facilities, personal hygiene facilities, and so forth. For present purposes such a shelter may also be equipped to provide for water treatment (such as filtering, salt removal, bacteria removal, and so forth), waste treatment and/or recycling, electrical power generation, and/or air treatment (including but not limited to conditioning, filtering, and so forth). By one approach such a floating life-sustaining facility based shelter can also be equipped with communication facilities including a variety of wireless broadcast capabilities, long-distance two-way communications capabilities, and so forth.

For a variety of reasons it may be appropriate to provide a plurality of floating life-sustaining facilities in various locations throughout a relatively wide geographic region. Such dispersal can aid with reducing the logistical difficulties of transporting authorized beneficiaries or life-sustaining resources to or from such facilities during times of need. Dispersed facilities can also help ensure that at least some will more likely survive civilly-catastrophic events that pose sufficient destructive force to destroy such a facility. Thus, the process 100 may optionally include substantially fixing 185 the floating life-sustaining facility at a predetermined location in anticipation of the occurrence of a civilly-catastrophic event. Fixing the facility may include, for example, anchoring the facility, providing global positioning information to allow the facility to move against drift to remain substantially fixed, tethering the facility to a fixed point, and the like.

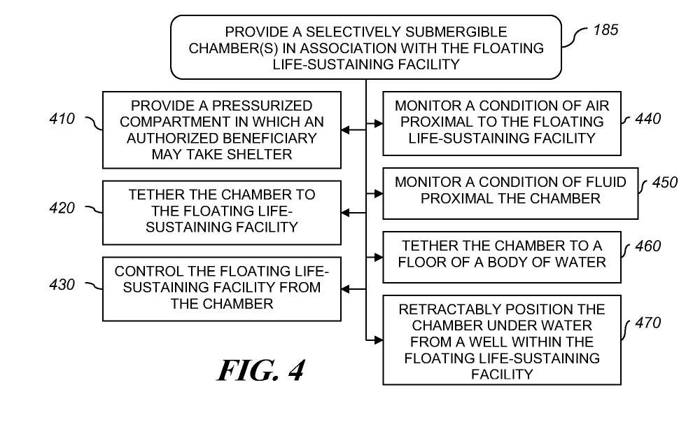

Furthermore, the floating life-sustaining facility may be provided with a variety of modifications and enhancements depending on its intended use. For instance, a floating life-sustaining facility may be provided 190 with a selectively submergible chamber in association with it. The selectively submergible chamber will be further described with reference to FIGS. 2 and 4. Like the floating life-sustaining facility 205 with which it is associated, the submergible chamber 220 or 225 may be provided with one or more of a variety of features. FIG. 2 illustrates two example submergible chambers, a chamber 225 used primarily for storage and a chamber 220 for occupation by authorized beneficiaries and attendants of the floating life-sustaining facility. Either may be provided with one or more of the features discussed herein though, for simplicity, subsequent reference will be limited to the submergible chamber 220.

These teachings contemplate that the submergible chamber 220 may be provided 410 with a pressurized compartment in which at least one authorized beneficiary 230 may take shelter wherein the selectively submergible chamber 220 is retractably positionable under water during a time of need. The selectively submergible chamber 220 may be as simple as a diving bell that may be used by an authorized beneficiary for a short period of time and that is available at a moment's notice. Such a chamber 220 may be selectively deployed from the deck of a ship or otherwise extendable into the water 235, and the chamber 220 will move with the floating life-sustaining facility 205 as the facility 205 moves.

By another approach, the submergible chamber 220 may be tethered 420 to the floating life-sustaining facility 205 wherein the selectively submergible chamber 220 may move vertically relative to the floating life-sustaining facility 205 when disposed within water 235 upon which the floating life-sustaining facility 205 is floating. In this approach, the method used to tether the chamber 220 to the floating life-sustaining facility 205 substantially maintains the vertical alignment of the chamber 220 relative to the facility. Such arrangements are known and may be applied in this provision of the chamber 220, such as providing vertical rail(s) secured to the facility 205, along which the selectively submergible chamber 220 may travel.

Optionally, the submergible chamber 220 may control 430, at least in part, the floating life-sustaining facility 205. Such an arrangement may be advantageous where, for safety reasons, the authorized beneficiary 230 should remain below water for a relatively long period of time. In such situations, having control over the floating life-sustaining facility 205 would allow the occupants of the chamber 220 to control where the floating life-sustaining facility 205 goes and its operations. Non-limiting examples of how the floating life-sustaining facility 205 may be controlled 430 from the selectively submergible chamber 220 include controlling propulsion, navigation, security, lighting, communications, movement of the selectively submergible chamber, defensive systems, and life support. A control panel 235, for example, may be provided in the chamber 220 to control the floating life-sustaining facility 205.

An additional feature that the submergible chamber 220 may control is the ability to monitor 440 at least one condition of air 240 proximal to the floating life-sustaining facility 205. Various known sensors, generally shown as element 245, disposed on or about the floating life-sustaining facility 205 are able to monitor conditions such as temperature, pressure, light level, humidity, wind speed and direction, contamination level, and/or a level of ionizing radiation, for example. These sensors 245 may be monitored from the chamber 220 to inform those in the chamber 220 as to the conditions above the fluid 250, typically water.

Similarly, the submergible chamber 220 may monitor 450 a condition of fluid proximal the selectively submergible chamber 220. Various known sensors, generally shown as element 255, disposed on or about the chamber 220 are able to monitor conditions such as temperature, pressure, light level, fluid flow speed and direction, contamination level, and/or a level of ionizing radiation, for example. These sensors 255 may be monitored from the chamber 220 to inform those in the chamber 220 as to the conditions of the fluid 250. By monitoring the air and fluid conditions, those in the chamber 220 may make decisions regarding whether and where to move the chamber 220 and/or the floating life-sustaining facility 205, when to move to the floating life-sustaining facility 205, and the like.

Another option for the chamber 220 includes configuring and arranging the chamber 220 to be selectively tethered 460 to a floor 260 of a body of water 250. In this manner, the chamber 220 and, if tethered to the floating life-sustaining facility 205, the facility 205 may be held in a relatively stationary place. The chamber 220 may be tethered to the floor 260 by one or more anchors or other known means for attachment to a sea floor 260.

As an additional optional feature, the selectively submergible chamber 220 may be retractably positionable under water 250 during a time of need from a well 265 within the floating life-sustaining facility 205. By disembarking from a well 265, the chamber 220 may be more easily loaded and unloaded or boarded and unboarded. Also, the chamber 220 may be more easily retracted and drag on the floating life-sustaining facility may be reduced if the chamber 220 moves into and from a well 265.

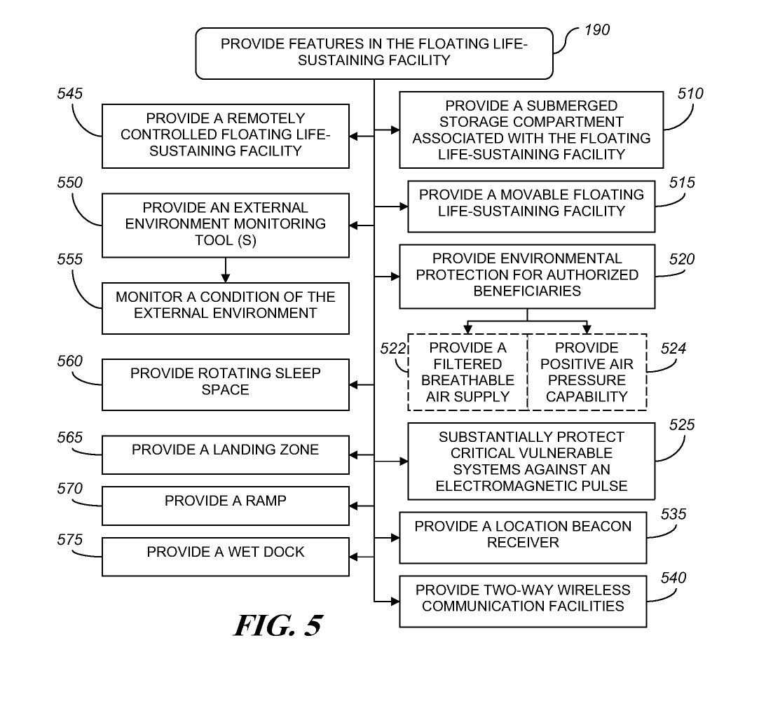

As such, the well 265 is one example of various features that may be provided 195 in association with a floating life-sustaining facility. Further examples of such features will be described with reference to FIGS. 2 and 5. For instance, the floating life-sustaining facility 205 or 210 may be provided 510 with a submerged storage compartment 270 or 275 that is associated with the floating life-sustaining facility 205 or 210. The storage compartment 270 or 275 may be part of or an entire selectively submergible chamber 225. By another approach, the compartment 275 may be a submerged portion of a floating life-sustaining facility 210; for example, where a floating life-sustaining facility 210 comprises a buoy, an underwater portion of the buoy may be the compartment 275 that stores life-sustaining resources, survival supplies, and/or non-necessities. By another approach, the compartment 277 may be stored on a sea floor 260 in an area typically occupied by an associated floating life-sustaining facility 205 such that those occupying the floating life-sustaining facility 205 may retrieve supplies stored in the compartment 277 as needed. By being submerged, the supplies will typically be protected from various conditions that may damage the supplies such as against ionizing radiation and the like.

Typically, these teachings will accommodate providing 515 a movable floating life-sustaining facility 205. Example movable floating life-sustaining facilities 205 include a hovercraft, an amphibious vehicle, a barge, a pilotless craft, and a remotely piloted craft. These facilities 205 may be powered in any conventional way including by propeller 280, sail 285, jet (not illustrated), paddle (not illustrated), and/or the like. Non-movable examples include a buoy 210 or a tethered storage device 287, such as a device that floats, but is tethered to the sea floor 260, pier, or other structure so as to stay submerged on station for a substantial period of time.

Another option is to provide 520 the floating life-sustaining facility 205 with, at least in part, environmental protection for authorized beneficiaries of the subscriptions. Examples of such environmental protection include providing 522 a filtered, breathable air supply and providing 524 positive air pressure capability to discourage airborne contaminants from entering the floating life-sustaining facility 205. Similarly, an air lock may be provided as part of the floating life-sustaining facility 205. Example airborne contaminates include radiological, biological, and chemical contaminants.

Yet another option to protect the floating life-sustaining facility 205 includes substantially protecting 525 the critical vulnerable systems of the floating life-sustaining facility against an electromagnetic pulse. Known protection systems include providing the critical vulnerable systems within Faraday cages. Such protection may help ensure the operation of electronic systems within the floating life-sustaining facility 205 in the event of being exposed to a damaging electromagnetic pulse.

Another optional feature for the floating life-sustaining facility includes providing 535 a location beacon receiver in association with the floating life-sustaining facility 205. The location beacon receiver may assist authorized beneficiaries or attendants in locating the floating life-sustaining facility 205 in times of need. The location beacon receiver optionally may be operated at all times or only upon the occurrence of a civilly-catastrophic event. Another approach to communicating with the floating life-sustaining facility includes providing 540 the floating life-sustaining facility 205 with two-way wireless communication facilities. Such communication facilities are known and may be employed.

The provision of two-way wireless communication can enable a further option that includes providing 545 a remotely controlled floating life-sustaining facility. With such a feature, the floating life-sustaining facility 205 may be left unattended until a need for the facility 205 arises. Then, if the facility 205 is needed and an attendant cannot reach it, the facility 205 may be remotely piloted to the place of need or away from a threat.

As discussed above in connection with the selectively submergible chamber 220, these teachings contemplate providing 550 the floating life-sustaining facility 205 with at least one external environment monitoring tool. Example external environment monitoring tools include air condition measurement devices as known in the art, a periscope, a video transmission as may be provided by video cameras, a captured still image transmission, a local sensor, a window, a sonar system, and a porthole. Thus, one may use the external environment monitoring tool(s) to monitor 555 at least one condition of the external environment, such as a threat level, temperature, pressure, light level, humidity, wind speed and direction, contamination level, and/or a level of ionizing radiation, for example.

Optionally, the floating life-sustaining facility 205 may be configured to provide 560 rotating sleep space such that more people may inhabit the floating life-sustaining facility 205 than a number of beds as are available in the floating life-sustaining facility 205. So configured, the floating life-sustaining facility 205 may shelter a larger number of authorized beneficiaries during a time of need.

With reference to FIGS. 2, 3, and 5, another option includes providing 565 a landing zone 310 as part of the floating life-sustaining facility 205. The landing zone 310 may include a number of configurations. For example, the landing zone 310 may include an area 320 for helicopters to land. A similar configuration may include facilities sufficient for airlifting of supplies. If the floating life-sustaining facility 205 included sufficient deck space, the landing zone 310 may include facilities, such as a landing strip 330, for the take off and landing of aircraft. The landing zone 310 may provide the benefit of having several ways to move supplies and/or people onto or off of the floating life-sustaining facility 205.

Another possible configuration includes providing 570 a ramp 290 extending from the floating life-sustaining facility 205 to water level. The ramp 290 can be provided, for example, for the launch and retrieval of hovercraft, water craft, a hydrofoil, an amphibious vehicle, a hovercraft, a rigid inflatable boat, and other vehicles. Similarly, the ramp 290 may facilitate access to the water for people on the floating life-sustaining facility 205. The ramp 290 is preferably collapsible and/or retractable to facilitate movement of the floating life-sustaining facility 205. The ramp 290 may also be included with the landing zone 310 to further facilitate movement of people and/or vehicles.

Another option for providing access to and from the floating life-sustaining facility 205 is to provide 575 a wet dock 340 for accepting arrival of water craft 350 within the floating life-sustaining facility 205. Wet docks 340 can be provided along the hull of a floating life-sustaining facility 205 to increase access for smaller water craft 350 to the facility 205. Optionally, the wet docks 340 may be closed to selectively increase security.

Thus configured, an acceptor of civil security subscriptions can provide shelter and/or resource storage with, typically, increased reliability and/or security. Selective placement of floating life-sustaining facilities can serve authorized beneficiaries near any coastline or shore and decrease the likelihood of interference with the provision of the shelter and/or resources through the discovery of unauthorized personnel.

Those skilled in the art will recognize that a wide variety of modifications, alterations, and combinations can be made with respect to the above described embodiments without departing from the spirit and scope of the invention. As but one example in this regard, floating life-sustaining facilities can be provided in any sufficiently large body of fluid, although bodies of water provide the most readily available locations. The bodies of fluid may be naturally occurring ponds, rivers, lakes, bays, seas, oceans, and the like or man-made collections of fluid or water. Also, any combination of the various features discussed herein may be applied to the facilities provided. Such modifications, alterations, and combinations are to be viewed as being within the ambit of the inventive concept.

I claim:

1. A method comprising:
providing a floating life-sustaining facility; and
accepting consideration-based private civil security subscriptions from subscribers with respect to selectively providing benefits for authorized beneficiaries of the subscriptions with respect to the floating life-sustaining facility in event of a civilly-catastrophic event.

2. The method of claim 1 wherein the floating life-sustaining facility further comprises at least one of the group comprising:
a floating shelter; and
a floating store of life-sustaining resources.

3. The method of claim 1 wherein providing the floating life-sustaining facility further comprises prepositioning the floating life-sustaining facility at a predetermined location.

4. The method of claim 3 wherein assessing a likelihood of at least one particular event occurring further comprises assessing a likelihood of interference with provision of the floating life-sustaining facility.

5. The method of claim 4 wherein prepositioning the floating life-sustaining facility at a predetermined location further comprises assessing a likelihood of at least one of the group comprising an occurrence of a particular event, a location of the at least one particular event, and interference caused by the particular event.

6. The method of claim 1 further comprising substantially maintaining the floating life-sustaining facility on station at least twelve miles away from any coast line of a sovereign entity.

7. The method of claim 1 further comprising substantially maintaining the floating life-sustaining facility on station in international waters outside the jurisdiction of any one sovereign nation.

8. The method of claim 1 wherein the floating life-sustaining facility is movable to a position outside of national territorial waters.

9. The method of claim 1 further comprising providing life-sustaining resources in a submerged storage compartment associated with the floating life-sustaining facility.

10. The method of claim 1 wherein the floating life-sustaining facility comprises a water craft.

11. The method of claim 10 wherein the water craft comprises at least one of:
a hovercraft;
a hydroplane;
a hydrofoil;
a ship;
a submarine;
an amphibious vehicle;
a barge;
a pilotless craft;
a single-hulled craft;
a double-hulled craft;
a triple-hulled craft;
a buoy;
a tethered storage device; and
a remotely piloted craft.

12. The method of claim 1 wherein the subscriptions differ from one another with respect to which benefits, accommodations, and features are accessible as per the subscription.

13. The method of claim 1 wherein the subscriptions comprise at least one of:
time-limited rights of access;
event-limited rights of access;
inheritable rights of access;
rights of access predicated upon a series of periodic payments;
rights of access predicated upon a one-time payment;
ownership-based rights of access;
non-transferable rights of access;
transferable rights of access;
membership-based rights of access;
fractionally-based rights of access;
non-ownership-based rights of access; and
option-based rights of access.

14. The method of claim 1 wherein the civilly-catastrophic event comprises at least one of:
a natural disaster; and
a non-naturally-caused disaster.

15. The method of claim 1 further comprising:
confirming identities of authorized beneficiaries gaining access to the floating life-sustaining facility.

16. The method of claim 15 wherein the identities of the authorized beneficiaries are confirmed by at least one of:
biometrics;
an identification card;
photographs;
visual confirmation;
an admission card;
a password;
a pass code;
a voice print; and
words.

17. The method of claim 15 further comprising:
upon confirmation of the authorized beneficiary's identity, automatically notifying at least one designated contact person in conjunction with the authorized beneficiary's entry into the floating life-sustaining facility.

18. The method of claim 1 further comprising:
maintaining the floating life-sustaining facility pending a need to permit subscription-based access to the floating life-sustaining facility upon occurrence of the civilly-catastrophic event.

19. The method of claim 1 further comprising:
providing survival items in the floating life-sustaining facility.

20. The method of claim 19 further comprising:
making adjustments to the survival items in the floating life-sustaining facility to reflect dynamically changing circumstances as occur during the consideration-based private civil security subscription.

21. The method of claim 19 wherein providing survival items comprises providing a plurality of life-sustaining resources as pertain to a plurality of differing categories of life's necessities.

22. The method of claim 21 wherein the plurality of life-sustaining resources comprise at least one of:
hydration resources;
nourishment resources;
shelter;
clothing;
security;
medical treatment; and
environmentally borne threat abatement.

23. The method of claim 19 wherein the survival items comprise at least one of:
consumable survival supplies; and
non-consumable survival supplies.

24. The method of claim 23 wherein the consumable survival supplies comprise at least one of:
food supplies;
potable water;
finite air supply;
emergency medical supplies;
batteries;
ammunition;
hygiene supplies;
sanitation supplies;
consumable fishing supplies;
signal flares;
sunscreen lotion;
anti-motion sickness medication;
insect repellant;
medications;
reverse osmosis water filters;
supplemental vehicular fuel supplies; and
spare parts for the floating life-sustaining facility.

25. The method of claim 23 wherein the non-consumable survival supplies comprise at least one of:
essential clothing items;
personal protection items;
bed linens;
beds;
food preparation items;
communications items;
power generation equipment;
repair and maintenance tools;
evacuation tools;
navigation equipment;
rescue tools and supplies;
radiation shielding;
biological/chemical agent protection;
spare vehicle parts;
self-rescue gear;
vehicular maintenance tools;
sails;
signal mirrors;
signal flags;
life rafts;
life boats;
rope;
life preservers;
fishing supplies;
vision enhancement devices;
semaphore materials;
deck shoes; and
weapons.

26. The method of claim 1 wherein providing the floating life-sustaining facility comprises providing a plurality of floating life-sustaining facilities.

27. The method of claim 26 further comprising physically coupling at least two of the floating life-sustaining facilities together.

28. The method of claim 26 further comprising physically coupling at least two of the plurality of floating life-sustaining facilities together such that a self-powered vehicle can simultaneously transport the at least two floating life-sustaining facilities.

29. The method of claim 26 wherein at least two of the floating life-sustaining facilities comprise different benefits, accommodations, and features from one another.

30. The method of claim 29 wherein the different benefits, accommodations, and features further comprise at least one of the group comprising:
a floating shelter having a larger selection of foods available relative to another floating shelter;
a floating shelter having a larger amount of authorized beneficiary privacy available relative to another floating shelter;
a floating shelter having a larger selection of non-necessities available relative to another floating shelter;
a floating shelter having a larger selection of consumable items available relative to another floating shelter;
a floating shelter having a larger selection of non-consumable items available relative to another floating shelter;
a floating shelter having increased security relative to another floating shelter;
a floating shelter having increased communications abilities available relative to another floating shelter;
a floating shelter having an increased availability of life sustaining resources relative to another floating shelter;
a floating shelter having higher quality living quarters relative to another floating shelter;
a floating shelter having a larger availability of health care options available relative to another floating shelter;
a floating shelter having a larger availability of educational materials available relative to another floating shelter; and
a floating shelter having a higher level of entertainment options available relative to another floating shelter.

31. The method of claim 30 wherein having a larger amount of authorized beneficiary privacy available further comprises having an increased amount of private space comprising at least one of the group comprising:
personal hygiene facilities;
personal storage space;
personal food preparation facilities;
personal external communications facilities;
personal entertainment space;
personal education space;
personal training space;
personal workshop space;
personal place to worship;
storage for at least one personalized item; and
personal meeting facilities.

32. The method of claim 31 wherein the at least one personalized item further comprises at least one of a group comprising:
specialized gear;
personal dietary requirements;
specialized clothing;
specialized safety gear;
specialized transport; and
personal medicine.

33. The method of claim 30 wherein the non-necessities further comprise at least one of the group comprising:
luxury consumable items;
luxury non-consumable items;
non-essential clothing;
non-human nourishment;
barter medium;
entertainment apparatus;
educational tools;
physical conditioning, exercise, and maintenance training and equipment;
crafts supplies and training;
towels;
bed linens;
food preparation items;
worship related items;
communication items; and
sports equipment and facilities.

34. The method of claim 30 wherein the consumable items further comprise at least one of the group comprising:
food supplies;
potable water;
medical supplies;
safety equipment; and
hygiene supplies.

35. The method of claim 30 wherein having an increased availability of life sustaining resources further comprises having an increased availability of at least one of the group comprising:
hydration resources;
nourishment resources;
shelter; and
environmentally borne threat abatement items.

36. The method of claim 30 wherein having higher quality living quarters further comprises having a higher quality of at least one of the group comprising:
sleeping accommodations;
food preparation facilities;
worship facilities;
personal hygiene facilities;
business activities facilities;
exercise facilities;
spa facilities; and
sitting accommodations.

37. The method of claim 30 wherein having a higher level of entertainment options available further comprises having at least one of the group comprising:
a larger selection of videos;
improved broadcast reception;
a larger selection of board games;
having access to live entertainment;
having a larger selection of arts and crafts materials;
having improved religious-activity related facilities;
a larger workshop space;
a larger selection of workshop tools; and
a larger selection of reading materials.

38. The method of claim 1 wherein the floating life-sustaining facility comprises, at least in part, environmental protection for authorized beneficiaries of the subscriptions.

39. The method of claim 38 wherein the environmental protection comprises a positive air pressure capability to discourage airborne contaminants from entering the floating life-sustaining facility.

40. The method of claim 39 wherein the airborne contaminants comprise at least one of:
radiological contaminants;
biological contaminants;
chemical contaminants.

41. The method of claim 38 wherein the environmental protection comprises a filtered breathable air supply.

42. The method of claim 38 wherein the environmental protection comprises an air lock.

43. The method of claim 1 wherein the floating life-sustaining facility comprises, at least in part, a controlled point of ingress and egress.

44. The method of claim 43 wherein the floating life-sustaining facility comprises, at least in part, at least one additional controlled point of ingress and egress.

45. The method of claim 43 wherein the point of ingress and egress comprises a sealable point of ingress and egress.

46. The method of claim 45 wherein the sealable point of ingress and egress comprises a door that opens inwardly.

47. The method of claim 45 wherein the sealable point of ingress and egress comprises a locking mechanism such that access to the floating life-sustaining facility is selectively controllable.

48. The method of claim 1 wherein providing a floating life-sustaining facility comprises providing a floating life-sustaining facility that has at least its critical vulnerable systems substantially protected against an electromagnetic pulse.

49. The method of claim 1 further comprising:
providing at least one non-necessity of human life in the floating life-sustaining facility.

50. The method of claim 49 wherein the at least one non-necessity of human life comprises at least one of:
a luxury consumable item;
a luxury non-consumable item;
non-human nourishment;
barter medium;
an entertainment apparatus;
an educational tool;
physical conditioning, exercise, and maintenance training and equipment;
crafts supplies and training; and
sports equipment and facilities.

51. The method of claim 1 wherein the floating life-sustaining facility comprises storage space.

52. The method of claim 1 further comprising:
providing at least one at least substantially full-time attendant at the floating life-sustaining facility.

53. The method of claim 52 wherein at least one at least substantially full-time attendant at the floating life-sustaining facility is available to facilitate operation of the floating life-sustaining facility in response to a civilly-catastrophic event.

54. The method of claim 1 further comprising providing at least one attendant at the floating life-sustaining facility upon the occurrence of a civilly-catastrophic event.

55. The method of claim 1 wherein the floating life-sustaining facility comprises a remotely-controlled floating life-sustaining facility.

56. The method of claim 1 further comprising substantially fixing the floating life-sustaining facility at a predetermined location in anticipation of the occurrence of a civilly-catastrophic event.

57. The method of claim 1 wherein the floating life-sustaining facility comprises a landing zone.

58. The method of claim 57 wherein the landing zone further comprises facilities sufficient for landing of aircraft.

59. The method of claim 57 wherein the landing zone further comprises facilities sufficient for airlifting of supplies.

60. The method of claim 57 wherein the landing zone further comprises a ramp to water level.

61. The method of claim 60 wherein the ramp facilitates launch and retrieval of at least one of the group comprising hovercraft and water craft.

62. The method of claim 1 wherein the floating life-sustaining facility comprises, at least in part, a location beacon receiver.

63. The method of claim 1 further comprising:
provisioning the floating life-sustaining facility with two-way wireless communications facilities.

64. The method of claim 1 further comprising:
providing a selectively submergible chamber in association with the floating life-sustaining facility.

65. The method of claim 64 wherein the selectively submergible chamber comprises a pressurized compartment in which at least one authorized beneficiary may take shelter wherein the selectively submergible chamber is retractably positionable under water during a time of need.

66. The method of claim 64 wherein the selectively submergible chamber is tethered to the floating life-sustaining facility wherein the selectively submergible chamber may move vertically relative to the floating life-sustaining facility when disposed within water upon which the floating life-sustaining facility is floating.

67. The method of claim 64 wherein the floating life-sustaining facility is controllable, at least in part, from the selectively submergible chamber.

68. The method of claim 67 wherein the floating life-sustaining facility is controllable, at least in part, from the selectively submergible chamber with respect to at least one of:
propulsion;
navigation;
security;
lighting;
communications;
movement of the selectively submergible chamber;
defensive systems; and
life support.

69. The method of claim 64 wherein the selectively submergible chamber is configured and arranged to be selectively tethered to a floor of a body of water.

70. The method of claim 64 wherein the selectively submergible chamber can monitor at least one condition of air proximal to the floating life-sustaining facility.

71. The method of claim 70 wherein the at least one condition is an ionizing radiation level.

72. The method of claim 64 wherein the selectively submergible chamber can monitor a condition of fluid proximal the selectively submergible chamber.

73. The method of claim 72 wherein the condition is an ionizing radiation level.

74. The method of claim 64 wherein the selectively submergible chamber is retractably positionable under water during a time of need from a well within the floating life-sustaining facility.

75. The method of claim 1 further comprising:
provisioning the floating life-sustaining facility with at least one external environment monitoring tool;
using the external environment monitoring tool to monitor at least one condition of the external environment.

76. The method of claim 75 wherein the at least one external environment monitoring tool comprises at least one of:
a periscope;
a video transmission;
a captured still image transmission;
a local sensor;
a window;
a sonar system; and
a porthole.

77. The method of claim 1 further comprising providing floating life-sustaining facility location information to authorized beneficiaries.

78. The method of claim 1 wherein providing the floating life-sustaining facility further comprises providing rotating sleep space such that more people may inhabit the floating life-sustaining facility than a number of beds as are available in the floating life-sustaining facility.

79. The method of claim 1 wherein the floating life-sustaining facility comprises a wet dock for accepting arrival of water craft within the floating life-sustaining facility.

80. The method of claim 1 wherein the floating life-sustaining facility comprises a ramp extending from the floating life-sustaining facility to water level.

81. The method of claim 80 wherein the ramp is provided for launching and receiving at least one of a group comprising:
a hydrofoil;
a water craft;
an amphibious vehicle;
a hovercraft; and
a rigid inflatable boat.
